(19) United States
(12) Patent Application Publication
(10) Pub. No.: US 2003/0166555 A1
(43) Pub. Date: Sep. 4, 2003

(54) Methods and compositions for regulating memory consolidation

(76) Inventors: Cristina M. Alberini, New York, NY (US); Mark F. Bear, Bristol, RI (US)

Correspondence Address: David E Brook Esq, Hamilton Brook Smith & Reynolds PC, 530 Virginia Road, PO Box 9133, Concord, MA, 01742-9133, US

(21) Appl. No.: 10/251,661

(22) Filed: Sep. 20, 2002

(30) Foreign Application Priority Data
Apr. 2, 2001 (WO) PCT/US01/10661

(51) Int. Cl. A61K 38/18; G01N 33/53

(52) U.S. Cl. 514/12; 435/7.1

(57) ABSTRACT

The present invention is based on the discovery of genes that are up- or down-regulated in inhibitory avoidance, e.g., long-term memory, which genes are therefor believed to have roles in memory consolidation. In particular, we have discovered that memory consolidation involves the regulation of expression of such genes as zif268 (EGR1), insulin-like growth factor (IGF-1), glutamate receptor 1 (GluR1), glutamate receptor 2 (GluR2), c/EBPβ and VGF. For ease of reading, these genes are collectively referred to herein as “LTM genes”, and their encoded proteins as “LTM proteins”.

### RELATED APPLICATION DATA

This application claims priority to U.S. Provisional Patent Application No. 60/193,614 filed on Mar. 31, 2000, and to International Application No. PCT/US01/10661, international filing date Apr. 2, 2001, both specifications of which are incorporated by reference herein.

### BACKGROUND OF THE INVENTION

Behavioral research has found that the human mind consolidates memory at certain key time intervals. The initial phase of memory consolidation occurs in the first few minutes after we are exposed to a new idea or learning experience. If a learning experience has on-going meaning to us, the next week or so serves as a further period of memory consolidation. In effect, in this phase, the material moves from short-term memory to long-term memory for storage.

It has been known for several decades that the formation of long-term memory requires gene expression. The prevailing hypothesis for the formation of long-term memory (LTM) is that introduction of a memory item alters the pattern of existing neuronal connectivity to form a neuronal network that will subserve the information for long-term storage. Modulation of synaptic efficacy is induced by changes in synaptic transmission within selected synapses or alteration in synaptic contacts. These changes are in turn supported by molecules that underlie transmission or synaptic remodeling. It is suggested that modulation of gene expression is needed for LTM formation to overcome the relative short lifetime of proteins in neurons (as compared with enduring memory).

In animal models of learning and memory, the requirement for de novo protein synthesis around the time of training has long been a definitive property of long-term memory that separates it from other types of memory retention. Thus, the storage of long-term memory is associated with cellular program(s) of gene expression, altered protein synthesis, and the growth of new synaptic connections. For instance, recent work suggests that this property of memory formation may have a specific molecular underpinning that involves cAMP-responsive transcription and that is mediated through the cAMP responsive element binding protein (CREB) family of transcription factors.

CREB is a nuclear protein that modulates the transcription of genes with cAMP responsive elements in their promoters. Increases in the concentration of either calcium or cAMP can trigger the phosphorylation and activation of CREB. Following its phosphorylation, CREB binds to the enhancer element CRE which is located in the upstream region of cAMP-responsive genes, thus triggering transcription. Some of the newly-synthesized proteins are additional transcription factors that ultimately give rise to the activation of late response genes, whose products are responsible for the modification of synaptic efficacy leading to LTM.

CREB subserves the formation of memories of various types of tasks that utilize different brain structures. Evidence is available suggesting that CREB regulates the transcription of genes that subserve LTM. In Aplysia, for example, CREB activation has been interfered with by microinjection of CRE containing oligonucleotides into cultured neurons. In Drosophila, CREB function has been disrupted using a reverse genetic approach. Thus, LTM has been specifically blocked by the induced expression of a CREB repressor isoform, and enhanced by the induced expression of an activator isoform. In mouse, the role of CREB has been confirmed by behavioural analyses of a knock-out line with a targeted mutation in the CREB gene. In these mutants, learning and short term memory are normal, whereas long term memory is impaired. On the whole, the data suggest that encoding of long term memories involve highly conserved molecular mechanisms.

Animals with lesions of the medial temporal lobes and related thalamic structures show a profound disruption of memory consolidation. We have previously demonstrated that fornix-dependent lesion-induced amnesia is associated with abnormal regulation of gene expression in specific subregions of the hippocampus. See, for example, Taubenfeld et al. (1999) Nat Neurosci 2:309-10. In normal animals, inhibitory avoidance training produces a rapid and persistent increase in the phosphorylation of CREB, which is a necessary step in the regulation of CRE-mediated gene expression required for memory consolidation. The change in CREB phosphorylation is largely confined to hippocampal fields CA1 and dentate gyrus, and lasts at least 6 hours after training. Animals with fornix lesions learn the inhibitory avoidance and display memory at control levels for up to 6 hours, however, by 24 hours they exhibit amnesia. The amnesic animals also fail to exhibit any increase in hippocampal CREB phosphorylation after training. Our results suggest that hippocampal inputs passing through the fornix regulate consolidation of this form of memory via regulation of CREB-mediated gene expression in hippocampal neurons.

Initial learning is likely to result from changes in the transmission of synapses conveying information about where the animal is in space. Whether or not these changes are made permanent depends on the timely occurrence of new gene expression. Signals impinging on hippocampal neurons via the fornix contribute to memory consolidation by modulating the gene expression required for the establishment of long-term memory. Identification of the critical chemical signals and their transduction pathways can suggest treatments for amnesia associated with damage to the temporal lobe memory system.

### SUMMARY OF THE INVENTION

One aspect of the present invention provides a a method for modulating long term memory consolidation in an animal comprising treating an animal with an agent that modulates the activity of one or more of zif 268, insulin-like growth factor, glutamate receptor 1 (GluR1), glutamate receptor 2 (GluR2), c/EBPβ and VGF.

Another aspect of the present invention relates to a method for enhancing long term memory consolidation in an animal comprising treating an animal with an agent that modulates a signal transduction pathway of glutamate receptor 1 (GluR1) or glutamate receptor 2 (GluR2), which agent is a ligand for the GluR1 or GluR2 receptor.

Still another aspect of the present invention relates to a method for identifying an agent (e.g., as a drug discovery assay) which modulates memory consolidation, comprising,

(i) providing a reaction system for detecting the activity of a product encoded by a gene selected from the group consisting of of zif 268, insulin-like growth factor, glutamate receptor 1 (GluR1), glutamate receptor 2 (GluR2), c/EBPβ and VGF;

(ii) contacting said system with a test compound; and

(iii) determining if the test compound alters the activity of the gene product.

Yet another asepect of the present invention provides a method for identifying an agent (e.g., as a drug discovery assay) which modulates memory consolidation, comprising,

(i) providing a reaction system for detecting the level of expression of a gene selected from the group consisting of of zif 268, insulin-like growth factor, glutamate receptor 1 (GluR1), glutamate receptor 2 (GluR2), c/EBPβ and VGF;

(ii) contacting said system with a test compound; and

(iii) determining if the test compound alters the level of expression of the gene.

In certain preferred embodiments, the reaction system is a cell-free system, such as a purified protein preparation or a cell-lysate. In other embodiments, the reaction system is a whole cell system.

In preferred embodiments, the assay can be used to identify agents which modulate memory consolidation from amongst a plurality of different test agents.

In certain preferred embodiments, the test compound can be small organic molecules, e.g., those having a molecular weight less than 2500 amu.

Still another aspect of the present invention provides a method of conducting a drug discovery business comprising:

(i) identifying, by one or more of the above drug discovery assay, a test compound which the level of expression of the gene or the activity of the gene product;

(ii) conducting therapeutic profiling of agents identified in step (i), or further analogs thereof, for efficacy and toxicity in animals; and

(iii) formulating a pharmaceutical preparation including one or more agents identified in step (ii) as having an acceptable therapeutic profile.

In certain preferred embodiments, the business method includes an additional step of establishing a distribution system for distributing the pharmaceutical preparation for sale, and may optionally include establishing a sales group for marketing the pharmaceutical preparation.

Yet another aspect of the present invention provides a method of conducting a target discovery business comprising:

(i) identifying, by one or more of the above drug discovery assay, a test compound which the level of expression of the gene or the activity of the gene product;

(ii) (optionally) conducting therapeutic profiling of agents identified in step (i), or further analogs thereof, for efficacy and toxicity in animals; and

(iii). licensing, to a third party, the rights for further drug development of said identified agents.

Still another aspect of the invention provides a a pharmaceutical preparation comprising, one or more compounds identified in the above assays, formulated in a pharmaceutically acceptable excipient. In certain embodiments, the preparation can also include one or more of a neuronal growth factor, a neuronal survival factor, and/or a neuronal tropic factor.

The subject pharmaceutical preparations can also include an agent that activates CREB-dependent transcription, such as a cAMP elevating agent, e.g., a cAMP agonist activates adenylate cyclase, cAMP analog, or a cAMP phosphodiesterase inhibitor.

The subject pharmaceutical preparations can be administered to an animal as part of a treatment method for enhancing memory consolidation in an animal, or otherwise enhancing the functional performance of CNS neurons.

Yet another aspect of the invention provides a method for assessing a patient for learning and/or memory functional performance including a step of detecting the expression of, or a mutation in, one or more genes selected from the group consisting of zif 268, insulin-like growth factor, glutamate receptor 1 (GluR1), glutamate receptor 2 (GluR2), c/EBPβ and VGF, or the level of activity of the gene products thereof, (optionally) in the patient's hippocampus.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1. Time-course Northern blot analysis of zif268 and c-fos following IA training. Increase in zif268, but not c-fos mRNA is evident in all animals at 9 and 20 hr after training.

FIG. 2. Time course Northern blot analysis of C/EBPβ and cyclophilin (control) mRNA following IA training in hippocampi of unoperated and fornix-lesion rats.

FIG. 3. Broad scale expression profiling with cDNA expression arrays. Side by side hybridizations with cDNA probes prepared from two different RNA populations allow the simultaneous comparison of the expression levels of all the cDNAs on the array. (from Clontech user manual).

FIG. 4. Examples of changes in gene array hybridizations reflecting differential expression of mRNAs following IA training. Hippocampi of control rats are compared to hippocampi of rats trained and sacrificed 9 hr later. Note that on these arrays each sequence is spotted in duplicate.

FIG. 5. A Northern blot test to confirm the levels of certain transcripts.

FIG. 6. Panel A. Mean escape latency for rats trained on the water maze. Rats received eight trials a day, for four consecutive days. Panel B. Mean escape latency on each of the eight trials of day one (trials 1-8) and day two (trials 9-16).

### DETAILED DESCRIPTION OF THE INVENTION

I. Overview

The present invention is based on the discovery of genes that are up- or down-regulated in inhibitory avoidance, e.g., long-term memory, which genes are therefor believed to have roles in memory consolidation. In particular, we have discovered that memory consolidation involves the regulation of expression of such genes as zif268 (EGR1), insulin-like growth factor (IGF-1), glutamate receptor 1 (GluR1), glutamate receptor 2 (GluR2), c/EBPβ and VGF. For ease of reading, these genes are collectively referred to herein as “LTM genes”, and their encoded proteins as “LTM proteins”.

One aspect of the present invention relates to the use of such genes and their products for carrying out assays designed to identify agents which, by modulating the function of one or more of the LTM genes or LTM proteins, can be used to modify long term memory consolidation in animals. As described in further detail below, test agents can be assessed in a cell-based or cell-free assay for ability to inhibit or potentiate the activity of an LTM protein, e.g., by modulating an enzymatic activity of the protein, modulating the half-life of the protein, modulating the interaction of the protein with other proteins, nucleic acids, carbohydrates or other biological molecules, modulating the cellular localization of the protein and the like.

Still another aspect of the invention relates to the use of compounds identified in the subject drug screening assays for altering (increasing or decreasing) the occurrence of learning and/or memory defects in an animal, and thus, altering the learning ability and/or memory capacity of the animal. As a result, the compounds of the present invention may be useful as therapeutic agents in memory impairment, e.g., due to toxicant exposure, brain injury, epilepsy, mental retardation in children and senile dementia, including Alzheimer's disease.

II. Definitions

For convenience, the meaning of certain terms and phrases employed in the specification, examples, and appended claims are provided below.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Complementary” sequences as used herein refer to sequences which have sufficient complementarity to be able to hybridize, forming a stable duplex.

The “CREB” family of proteins (sometimes referred to as the ATF family) is best known for the three members that can mediate cAMP-responsive transcription: CREB itself, CREM and ATF-1 (DeGroot et al. (1993) Mol Endocrinol 7:145-153). These basic-region, leucine-zipper proteins bind to DNA sequences, called cAMP response element (CRE) sites, which are often found in the upstream regulatory regions of genes whose synthesis is cAMP responsive. Molecular analysis has shown that CRE sites, and their interaction with CREB family members, are necessary for cAMP responsiveness. After the catalytic subunit of PKA translocates to the nucleus, it can directly phosphorylate the serine residue at position 133 on CREB, thus activating the protein and directly linking the cAMP transduction pathway to the induction of new gene expression (Backsai et al. (1993) Science 260: 222-226; and Hagiwara et al. (1993) Mol Cell Biol 13:4852-4859). CREB is also phosphorylated via other kinases, such as described above and in Deisseroth et al. (1996) Neuron 16:89-101; Impey et al. (1996) Neuron 16:973-82; and Impey et al. (1998) Neuron 21:869-883.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding” a polypeptide may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a protein with the same biological activity.

As used herein, the term “gene” or “recombinant gene” refers to a nucleic acid molecule comprising an open reading frame encoding a polypeptide, including both exon and (optionally) intron sequences. The term “intron” refers to a DNA sequence present in a given gene which is not translated into protein and is generally found between exons.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The term “interact” as used herein is meant to include detectable interactions between molecules, such as can be detected using, for example, a two hybrid assay. The term interact is also meant to include “binding” interactions between molecules. Interactions may be protein-protein or protein-nucleic acid in nature.

The term “isolated” as used herein with respect to nucleic acids, such as DNA or RNA, refers to molecules separated from other DNAs or RNAs, respectively, that are present in the natural source of the macromolecule. For example, an isolated nucleic acid encoding a particular polypeptide preferably includes no more than 10 kilobases (kb) of nucleic acid sequence which naturally immediately flanks the gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “modulation” as used herein refers to both upregulation, i.e., stimulation, and downregulation, i.e. suppression, of a response.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse. The term “chimeric animal” is used herein to refer to animals in which the recombinant gene is found, or in which the recombinant is expressed in some but not all cells of the animal. The term “tissue-specific chimeric animal” indicates that one of the recombinant genes is present and/or expressed or disrupted in some tissues but not others.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangably herein when referring to a gene product.

The term “recombinant protein” refers to a polypeptide of the present invention which is produced by recombinant DNA techniques, wherein generally, DNA encoding a polypeptide is inserted into a suitable expression vector which is in turn used to transform a host cell to produce the heterologous protein. Moreover, the phrase “derived from”, with respect to a recombinant gene, is meant to include within the meaning of “recombinant protein” those proteins having an amino acid sequence of a native protein, or an amino acid sequence similar thereto which is generated by mutations including substitutions and deletions (including truncation) of a naturally occurring form of the protein.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the recombinant genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of proteins.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the protein is disrupted.

As used herein, the term “transgene” means a nucleic acid sequence encoding s polypeptide or an antisense transcript thereto, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, (e.g. as intron), that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a proteins, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption is caused by human intervention, including both recombination and antisense techniques.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forts of expression vectors which serve equivalent functions and which become known in the art subsequently hereto.

III. Exemplary Embodiments

A. Nucleic Acids Encoding LTM Proteins

As described below, certain embodiments of the assays of the invention use isolated nucleic acids comprising nucleotide sequences encoding a protein involved in memory consolidation, which proteins will be herein referred to as “LTM” proteins or polypeptides. The term equivalent is understood to include nucleotide sequences encoding functionally equivalent LTM polypeptides or functionally equivalent peptides having an activity of an LTM protein such as described herein. Equivalent nucleotide sequences will include sequences that differ by one or more nucleotide substitution, addition or deletion, such as allelic variants.

Preferred nucleic acids are vertebrate LTM nucleic acids. Particularly preferred vertebrate LTM nucleic acids are mammalian. Regardless of species, particularly preferred LTM nucleic acids encode polypeptides that are at least 80% similar to an amino acid sequence of a vertebrate LTM protein. In one embodiment, the nucleic acid is a cDNA encoding a polypeptide having at least one bioactivity of the subject LTM polypeptide.

Still other preferred nucleic acids of the present invention encode an LTM polypeptide which is comprised of at least 2, 5, 10, 25, 50, 100, 150 or 200 amino acid residues. For example, preferred nucleic acid molecules for use as probes/primer or antisense molecules (i.e. noncoding nucleic acid molecules) can comprise at least about 6, 12, 20, 30, 50, 100, 125, 150 or 200 base pairs in length, whereas coding nucleic acid molecules can comprise about 300, 400, 500, 600, 700, 800, 900, 950, 975, 1000 base pairs.

Another aspect of the invention provides a nucleic acid which hybridizes under stringent conditions to a nucleic acid encoding a cloned LTM gene. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. Preferred nucleic acids have a sequence at least 75% homologous and more preferably 80% and even more preferably at least 85% homologous with an nucleic acid sequence of an LTM gene. Nucleic acids at least 90%, more preferably 95%, and most preferably at least about 98-99% homologous with a nucleic sequence of an LTM gene are of course also within the scope of the invention.

Nucleic acids having a sequence that differs from the nucleotide sequences shown in one of SEQ ID NOs: 1-X due to degeneracy in the genetic code are also within the scope of the invention. Such nucleic acids encode functionally equivalent peptides (i.e., a peptide having a biological activity of a LTM polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a LTM polypeptide. However, it is expected that DNA sequence polymorphisms that do lead to changes in the amino acid sequences of the subject LTM polypeptides will exist among mammalians. One skilled in the art will appreciate that these variations in one or more nucleotides (e.g., up to about 3-5% of the nucleotides) of the nucleic acids encoding polypeptides having an activity of a LTM polypeptide may exist among individuals of a given species due to natural allelic variation.

B. LTM Proteins

Certain assays of the present invention use isolated or recombinant LTM polypeptides which are isolated from, or otherwise substantially free of other cellular proteins, especially other signal transduction factors and/or transcription factors which may normally be associated with the LTM polypeptide. The term “substantially free of other cellular proteins” (also referred to herein as “contaminating proteins”) or “substantially pure or purified preparations” are defined as encompassing preparations of LTM polypeptides having less than about 20% (by dry weight) contaminating protein, and preferably having less than about 5% contaminating protein. Functional forms of the subject polypeptides can be prepared, for the first time, as purified preparations by using a cloned gene as described herein. By “purified”, it is meant, when referring to a peptide or DNA or RNA sequence, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substance, or solutions. In preferred embodiments, purified LTM preparations will lack any contaminating proteins from the same animal from which LTM is normally produced, as can be accomplished by recombinant expression of, for example, a human LTM protein in a non-human cell.

Full length proteins or fragments corresponding to one or more particular motifs and/or domains or to arbitrary sizes, for example, at least 5, 10, 25, 50, 75, 100, 125, 150 amino acids in length are within the scope of the present invention.

For example, isolated LTM polypeptides can be encoded by all or a portion of a nucleic acid sequence shown in any of SEQ ID NOs: 1-X. Isolated peptidyl portions of LTM proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an LTM polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) LTM protein.

Recombinant polypeptides preferred by the present invention, in addition to native LTM proteins, are encoded by a nucleic acid, which is at least 85% homologous and more preferably 90% homologous and most preferably 95% homologous with an amino acid sequence of an LTM protein. In a preferred embodiment, an LTM protein of the present invention is a mammalian LTM protein. It will be understood that certain post-translational modifications, e.g., phosphorylation and the like, can increase the apparent molecular weight of the LTM protein relative to the unmodified polypeptide chain.

Moreover, it will be generally appreciated that, under certain circumstances, it may be advantageous to provide homologs of one of the subject LTM polypeptides which function in a limited capacity as one of either an LTM agonist (mimetic) or an LTM antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of LTM proteins.

Homologs of each of the subject LTM proteins can be generated by mutagenesis, such as by discrete point mutation(s), or by truncation. For instance, mutation can give rise to homologs which retain substantially the same, or merely a subset, of the biological activity of the LTM polypeptide from which it was derived. Alternatively, antagonistic forms of the protein can be generated which are able to inhibit the function of the naturally occurring form of the protein, such as by competitively binding to a downstream or upstream member of the LTM cascade which includes the LTM protein. In addition, agonistic forms of the protein may be generated which are constitutively active. Thus, the LTM protein and homologs thereof provided by the subject invention may be either positive or negative regulators of memory consolidation.

C. Cells Expressing LTM Proteins

As described below, the assays of the invention may include cells transfected to express a recombinant form of the subject LTM polypeptides. The host cell may be any prokaryotic or eukaryotic cell. Thus, a nucleotide sequence derived from the cloning of mammalian LTM proteins, encoding all or a selected portion of the full-length proteir, can be used to produce a recombinant form of an LTM polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. MAP kinase, p53, WT1, PTP phosphatases, SRC, and the like. Similar procedures, or modifications thereof, can be employed to prepare recombinant LTM polypeptides by microbial means or tissue-culture technology in accord with the subject invention.

D. Drug Screening Assays

According to one aspect of the present invention, LTM genes and/or LTM gene products are used for carrying out assays designed to identify agents which, by modulating the function of one or more of the LTM genes, can be used to modify long term memory consolidation in animals. As described in further detail below, test agents can be assessed in a cell-based or cell-free assay for ability to inhibit or potentiate the activity of an LTM protein. As described in the examples, the LTM genes can range from cell surface receptors and secreted proteins to transcription factors. Accordingly, the invention contemplates such drug-screening formats which detect compounds that, e.g., modulate an enzymatic activity of the LTM protein, modulate the half-life of the LTM protein, modulate the interaction of the LTM protein with other proteins, nucleic acids, carbohydrates or other biological molecules, modulate the cellular localization of the LTM protein and the like. A variety of assay formats will suffice and, in light of the present inventions, will be comprehended by a skilled artisan.

Exemplary agents which can be tested in the subject drug screening assays include small organic molecules, e.g., having a molecular weight less than 2500 amu, more preferably less than less than 1000, 750 or 500 amu. Such molecules can include peptide and non-peptide moieties, nucleic acids, carbohydrates and the like. In many embodiments, it will be desirable to repeat the assay for a plurality of different test agents. For example, the subject assays can be repeated for at least 10 different test agents, and in other embodiments, for at least 100, or even at least 1000 different test agents.

(i) Cell-Free Assays

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements or with intrinsic enzymatic activity. Many of the LTM proteins identified by the subject method will be amenable to some form of cell-free assay formats. Soluble proteins, be they cytoplasmic or extracellular, can be recombinantly expressed and at least partially purified, or provided as lysates, for use in cell-free assays. Membrane-associated proteins can, in certain instances, be purified in detergent or liposomes, or isolated as part of a cell membrane fraction or organelle preparation.

Accordingly, in an exemplary screening assay of the present invention, a reaction mixture is generated including the LTM protein and one or more proteins (or nucleic acids) which interact with the LTM protein, such molecules being referred to herein as “LTM-interacting partners” or “LTM-IP”. Examples of LTM-IP include proteins that function upstream (including both activators and repressors of LTM activity), and proteins or nucleic acids which function downstream of the LTM polypeptide, whether they are positively or negatively regulated by it. The reaction mixture also includes one or more test compounds. Detection and quantification of complexes of the LTM protein with upstream or downstream LTM-IP provide a means for determining a compound's efficacy at inhibiting or potentiating complex formation between LTM and the LTM-IPs. The efficacy of the compound can be assessed by generating dose response curves from data obtained using various concentrations of the test compound. Moreover, a control assay can also be performed to provide a baseline for comparison. In one control assay, isolated and purified LTM polypeptide is added to a composition containing the LTM-IP, and the formation of a complex is quantitated in the absence of the test compound.

Complex formation between the LTM polypeptide and a binding partner may be detected by a variety of techniques. Modulation of the formation of complexes can be quantitated using, for example: detectably labeled proteins such as radiolabeled, fluorescently labeled, or enzymatically labeled proteins; by immunoassay; or by chromatographic detection.

Typically, it will be desirable to immobilize either LTM or its interacting partner to facilitate separation of complexes from uncomplexed forms of one or both of the proteins, as well as to accommodate automation of the assay. Binding of the LTM protein to an upstream or downstream element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/LTM (GST/LTM) fusion proteins can be adsorbed onto glutathione sepharose beads (Sigma Chemical, St. Louis, Mo.) or glutathione derivatized microtitre plates, which are then combined with a cell lysate or other preparation including the LTM-IP and the test compound, and the mixture incubated under conditions conducive to complex formation (in the absence of the test compound), e.g. at physiological conditions for salt and pH, though slightly more stringent conditions may be desired. Following incubation, the beads are washed to remove any unbound LTM-IP, and the matrix immobilized and the amount of LTM-IP in the matrix determined, or in the supernatant after the complexes are subsequently dissociated. Alternatively, the complexes can be dissociated from the matrix, separated by SDS-PAGE, and the level of LTM-IP found in the bead fraction quantitated from the gel using standard electrophoretic techniques.

Other techniques for immobilizing proteins or nucleic acids on matrices are also available for use in the subject assay. For instance, either LTM or its cognate binding partner can be immobilized utilizing conjugation of biotin and streptavidin. For instance, biotinylated LTM proteins can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the LTM protein, but which do not interfere with binding of upstream or downstream binding partners, can be derivatized to the wells of the plate, and the LTM protein trapped in the wells by antibody conjugation. As above, preparations of an LTM-IP and a test compound are incubated in the LTM-presenting wells of the plate, and the amount of complex trapped in the well can be quantitated. Exemplary methods for detecting such complexes, in addition to those described above for the GST-immobilized complexes, include immunodetection of complexes using antibodies reactive with the LTM binding partner, or which are reactive with the LTM protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion, protein with an LTM-IP. To illustrate, the LTM-IP can be chemically cross-linked or genetically fused with horseradish peroxidase, and the amount of polypeptide trapped in the complex can be assessed with a chromogenic substrate of the enzyme, e.g. 3,3′-diaminobenzadine tetrahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J. Biol. Chem. 249:7130).

For processes which rely on immunodetection for quantitating one of the proteins trapped in the complex, antibodies against the protein, such as anti-LTM antibodies, can be used. Alternatively, the protein to be detected in the complex can be “epitope tagged” in the form of a fusion protein which includes a second polypeptide sequence for which antibodies are readily available (e.g. from commercial sources). For instance, the GST fusion proteins described above can also be used for quantification of binding using antibodies against the GST moiety. Other useful epitope tags include mycepitopes (e.g., see Ellison et al. (1991) J. Biol. Chem. 266:21150-21157) which includes a 10-residue sequence from c-myc, as well as the pFLAG system (International Biotechnologies, Inc.) or the pEZZ-protein A system (Pharmacia, N.J.).

Other cell-free ebodiments include assays which detect an intrinsic activity of an LTM protein or a complex including an LTM protein, and identify compounds that increase or inhibit that activity. For instance, the reaction micture can be generated to include the LTP protein, a substrate for an enzymatic activity of the LTM protein, and the test agent. The rate of conversion of the substrate to product is determined, and can be compared to such control samples as the LTM proteins and substrate admixed alone. Test agents which are inhibitors of the LTM activity will decrease the rate of conversion of the substrate to product, whereas test agents that increase that rate are likely to be agonists of the LTM activity.

In preferred embodiments, the substrate is readily detectable, e.g., the conversion of substrate to product a colorimetric or fluorometric change in the reaction mixture which is detectable by spectroscopic means, or creates or destroys an epitope which is detectable by immunoassay.

(ii) Cell Based Assays

In addition to cell-free assays, such as described above, the readily available LTM proteins provided by the present invention also facilitates the generation of cell-based assays for identifying small molecule agonists/antagonists and the like. The ability of a test agent to alter the activitiy of an LTM protein in the cell may include directly detecting the formation of complexes including the LTM protein, detecting an intrinic enzymatic activity of the LTM protein, directly detecting a change in cellular localization of the LTM protein, detecting a post-translational modification to the LTM protein or a change in the stability of the LTM protein, or detecting the downstream consequence of any one of such events.

Such assays can be simple binding assays. For instance, where the LTM protein is a receptor, the assay can be used to identify compounds which bind to the receptor or effect the ability of the receptor to bind its ligand. In other embodiments, cells which are phenotypically sensitive to the presence or activity of the LTM protein, e.g., if it produces a morphological change in the cell, can be caused to over- or under-express a recombinant LTM protein in the presence and absence of a test agent of interest, with the assay scoring for modulation in LTM responses by the target cell which mediated by the test agent. As with the cell-free assays, agents which produce a statistically significant change in LTM-dependent responses (either inhibition or potentiation) can be identified. For example, the level of expression of genes or gene products which are up- or down-regulated in response to the presence or activity of an LTM protein can be detected. In preferred embodiments, the regulatory regions of such genes, e.g., the 5′ flanking promoter and enhancer regions, are operably linked to a detectable marker (such as luciferase) which encodes a gene product that can be readily detected.

In the event that the LTM proteins themselves, or in complexes with other proteins, are capable of binding DNA and modifying transcription of a gene, a transcriptional based assay could be used, for example, in which an LTM-responsive regulatory sequence is operably linked to a detectable marker gene.

In yet another aspect of the invention, the subject drug screening assays can utilized the LTM proteins to generate a “two hybrid” assay (see, for example, U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and PCT Publication No. WO 94/10300). Briefly, the two hybrid assay relies on reconstituting in vivo a functional transcriptional activator protein from two separate fusion proteins. In particular, the method makes use of chimeric genes which express hybrid proteins. To illustrate, a first chimeric gene can be generated with the coding sequence for a DNA-binding domain of a transcriptional activator fused in frame to the coding sequence for an LTM protein. The second hybrid protein encodes a transcriptional activation domain fused in frame to another polypeptide, e.g., and LTM-IP, which binds to the LTM protein. If the two fusion proteins are able to interact, e.g., form an LTM-dependent complex, they bring into close proximity the two domains of the transcriptional activator. This proximity is sufficient to cause transcription of a reporter gene which is operably linked to a transcriptional regulatory site which is bound by the DNA-binding domain of the first fusion proteins, and expression of the reporter gene can be detected and used to score for the interaction of the LTM and sample proteins.

a. Exemplary LTM Proteins: GluR1 and GluR2

In certain embodiments, the subject assays are used to identify compouds which modulate the activity of a glutamate receptor, such as GluR1 (SEQ ID Nos. 1 and 2) or GluR2 (SEQ ID Nos. 3 and 4).

The subject assays can be used to identifying agents which bind to the receptors and, e.g., mimic or potentiate the activity of the natural ligand, or which inhibit binding or signal transduction by the receptor. To illustate, for binding studies, test agents can be tested for competition with binding. For example, [³H]AMPA binding can be assessed as follows: cells expressing GluR1 or GluR2 are preincubated in 50 mM Tris-HCl buffer, pH 7.4, for 30 min, and then incubated at 4° C. for 4 h in 100 mM buffer plus 100 mM KCSN, 70 nM [³H]AMPA (53 Ci/mmol, NEN, Boston, Mass.) and the test agent. Binding of the radiolabeled AMPA is assessed and compared to the level of binding in the absence of the test compound. Nonspecific binding is measured in the presence of 1 mM L-glutamate.

The GluR1 and GluR2 receptors appears to be coupled to postsynaptic inositol phosphate metabolism, e.g., activating the PLC/PKC/CREB/MapK pathway, as well as cAMP-mediated pathways. Accordingly, generation of second messengers, and regulation of gene expression can be used to asses the ability of a test agent to inhibit or potentiate the activity of GluR1 or GluR2.

b. Exemplary LTM Proteins: Insulin-Like Growth Factor 1

IGF1 (SEQ ID No.s 7 and 8) is an extracellular protein which mediates its inductive effect on cells at least in part through binding to the IGF1 receptor (IGF1-R). The receptor has an intrinsic tyrosine kinase activity, which can be targeted in the subject assay for identifying inhibitors of the kinase activity.

Signal transduction from the receptor includes interaction with a series of proteins containing Src homology-2 (SH2) domains, including SH2-containing protein tyrosine phosphatase 2 (SHP-2) and SHC. The subject assay can be used to identify agents which inhibit the interaction of IGF1-R with SH2 domains, or which inhibit the enzymatic activity of an SH2 protein which interacts with IGF1-R. For instance, the assay can combine the tyrosine-phosphorylated IGF1-Rβ-subunit, at least the SH2 domain of SHP-2, and a test agent, and detect the ability of the test agent to alter the formation of complexes containing IGF1-R and SHP-2.

IGF1-R also signals through acitvation of the Jak protein tyrosine kinase family, with resultant phosphorylation of the STAT transcriptional activator factors. See, for example, Frank et al (1995) PNAS 92:7779-7783; Scharfe et al. (1995) Blood 86:2077-2085; Bacon et al. (1995) PNAS 92:7307-7311); and Sakatsume et al (1995) J. Biol Chem 270:17528-17534. Events downstream of Jak phosphorylation have also been elucidated. For example, IGF1-R can cause the phosphorylation of signal transducers and activators of transcription (STAT) proteins STAT1□, STAT2β, and STAT3, as well as of two STAT-related proteins, p94 and p95. The STAT proteins translocate to the nucleus and bind to specific DNA sequences, thus suggesting a mechanism by which IGF1-R may activate specific genes

Detection means which may be scored for in the present assay, in addition to direct detection of second messangers, such as by changes in phosphorylation, includes reporter constructs or indicator genes which include transcriptional regulatory elements responsive to the STAT proteins.

c. Exemplary LTM Proteins: Neuroendocrine VGF

The neurotropin-inducible gene VGF (SEQ ID Nos. 11 and 12) is expressed in neuronal and endocrine tissues. It encodes a secretory protein that is proteolytically processed in neuronal cells to low molecular mass polypeptides. In addition to targeting the second messanger induction caused by contacting cells with VGF, the subject assays can also be used to identify agents which inhibit the proteolytic processing of VGF.

d. Exemplary LTM Proteins: ZIF268

Zif268 (SEQ ID Nos. 5 and 6) is also known as krox-24, egr-1, TIS 8, NGFI-A or zenk. It is a zinc-finger transcription factor which binds to a so-called “EGR1 motif”, e.g., a trascriptional regulatory sequence of 5′-CGCCCCCGC or 5′-GCGTGGGCG. See, for example, Rauscher et al. (1990) Science 250:1259; and Pavletich et al. (1991) Science 252:809.

In certain embodiments, the subject assay can be a transcription based assay including a reporter gene having an EGR1 motif as part of its transcriptional regulatory sequences. Test agents can be assessed for their ability to enhance or inhibit Zif268-dependent transcription.

In other embodiments, test agents can be tested for their ability to ehance or inhibit binding of Zif268 with its EGR1 motif in a competitive binding assay, e.g, a nucleic acid including the motif.

e. Exemplary LTM proteins: C/EBPβ

In another embodiment, the target for the subject drug screening assay is the transcription factor C/EBPβ (SEQ ID Nos. 9 and 10), also referred to as the CCAAT/enhancer protein. The CCAAT/Enhancer Binding Protein (C/EBP) family belongs to the basic leucine zipper class of transcription factors. The C/EBP protein binds to the CCAAT-box (consensus GG^{T}/_{C}CAATCT).

As above with Zif268, C/EBP-dependent transcriptional activity or competition binding assays can be used to assessed the ability of test compounds to enhance or inhibit C/EBPβ activity.

f. Exemplary Screening and Selection Assays: Second Messenger Generation

When screening for bioactivity of test compounds, intracellular second messenger generation can be measured directly. A variety of intracellular effectors have been identified as being regulated by certain of the LTM proteins described above, including adenylyl cyclase, cyclic GMP, phosphodiesterases, phosphoinositidase C, and phospholipase A₂, as well as a variety of ions.

In one embodiment, the detection signal is provided by detecting phosphorylation of intracellular proteins, e.g., MEKKs, MEKs, or Map kinases, or by the use of reporter constructs or indicator genes which include transcriptional regulatory elements responsive to c-fos and/or c-jun.

In another embodiment, the GTPase enzymatic activity by G proteins can be measured in plasma membrane preparations by determining the breakdown of □³²P GTP using techniques that are known in the art (For example, see Signal Transduction: A Practical Approach. G. Milligan, Ed. Oxford University Press, Oxford England). When receptors that modulate cAMP are tested, it will be possible to use standard techniques for cAMP detection, such as competitive assays which quantitate [³H]cAMP in the presence of unlabelled cAMP.

Certain of the subject LTM proteins stimulate the activity of phospholipase C which stimulates the breakdown of phosphatidylinositol 4,5, bisphosphate to 1,4,5-IP3 (which mobilizes intracellular Ca++) and diacylglycerol (DAG) (which activates protein kinase C). Inositol lipids can be extracted and analyzed using standard lipid extraction techniques. DAG can also be measured using thin-layer chromatography. Water soluble derivatives of all three inositol lipids (IP1, IP2, IP3) can also be quantitated using radiolabelling techniques or HPLC.

The other product of PIP2 breakdown, DAG can also be produced from phosphatidyl choline. The breakdown of this phospholipid in response to receptor-mediated signaling can also be measured using a variety of radiolabelling techniques.

The activation of phospholipase A2 can easily be quantitated using known techniques, including, for example, the generation of arachadonate in the cell.

In the case of certain receptors, such as IGF1-R, it may be desirable to screen for changes in cellular phosphorylation. Such assay formats may be useful when the receptor of interest is a receptor kinase or phosphatase. For example, immunoblotting (Lyons and Nelson (1984) Proc. Natl. Acad. Sci. USA 81:7426-7430) using anti-phosphotyrosine, anti-phosphoserine or abti-phosphothreonine antibodies. In addition, tests for phosphorylation could be also useful when the receptor itself may not be a kinase, but activates protein kinases or phosphatase that function downstream in the signal transduction pathway.

One such cascade is the MAP kinase pathway that appears to mediate both mitogenic, differentiation and stress responses in different cell types. Stimulation of growth factor receptors results in Ras activation followed by the sequential activation of c-Raf, MEK, and p44 and p42 MAP kinases (ERK1 and ERK2). Activated MAP kinase then phosphorylates many key regulatory proteins, including p90RSK and Elk-1 that are phosphorylated when MAP kinase translocates to the nucleus. Homologous pathways exist in mammalian and yeast cells. For instance, an essential part of the S. cerevisiae pheromone signaling pathway is comprised of a protein kinase cascade composed of the products of the STE11, STE7, and FUS3/KSS1 senes (the latter pair are distinct and functionally redundant). Accordingly, phosphorylation and/or activation of members of this kinase cascade can be detected and used to quantitate receptor engagement. Phosphotyrosine specific antibodies are available to measure increases in tyrosine phosphorylation and phospho-specific antibodies are commercially available (New England Biolabs, Beverly, Mass.).

In yet another embodiment, the signal transduction pathway of the LTM protein upregulates expression or otherwise activates an enzyme which is capable of cleaving a substrate which can be added to the cell. The signal can be detected by using a detectable substrate, in which case lose of the substrate signal is monitored, or altenatively, by using a substrate which produces a detectable product. In preferred embodiments, the conversion of the substrate to product by the activated enzyme produces a detectable change in optical characteristics of the test cell, e.g., the substrate and/or product is chromogenically or fluorogenically active. In an illustrative embodiment the, signal transduction pathway causes a change in the activity of a proteolytic enzyme, altering the rate at which it cleaves a substrate peptide (or simply activates the enzyme towards the substrate). The peptide includes a fluorogenic donor radical, e.g., a fluorescence emitting radical, and an acceptor radical, e.g., an aromatic radical which absorbs the fluorescence energy of the fluorogenic donor radical when the acceptor radical and the fluorogenic donor radical are covalently held in close proximity. See, for example, U.S. Ser. Nos. 5,527,681, 5,506,115, 5,429,766, 5,424,186, and 5,316,691; and Capobianco et al. (1992) Anal Biochem 204:96-102. For example, the substrate peptide has a fluorescence donor group such as 1-aminobenzoic acid (anthranilic acid or ABZ) or aminomethylcoumarin (AMC) located at one position on the peptide and a fluorescence quencher group, such as lucifer yellow, methyl red or nitrobenzo-2-oxo-1,3-diazole (NBD), at a different position near the distal end of the peptide. A cleavage site for the activated enzyme will be diposed between each of the sites for the donor and acceptor groups. The intramolecular resonance energy transfer from the fluorescence donor molecule to the quencher will quench the fluorescence of the donor molecule when the two are sufficiently proximate in space, e.g., when the peptide is intact. Upon cleavage of the peptide, however, the quencher is separated from the donor group, leaving behind a fluorescent fragment. Thus, activation of the enzyme results in cleavage of the detection peptide, and dequenching of the fluorescent group.

In still other embodiments, the detectable signal can be produced by use of enzymes or chromogenic/fluorscent probes whose activities are dependent on the concentration of a second messanger, e.g., such as calcium, hydrolysis products of inositol phosphate, cAMP, etc. For example, the mobilization of intracellular calcium or the influx of calcium from outside the cell can be measured using standard techniques. The choice of the appropriate calcium indicator, fluorescent, bioluminescent, metallochromic, or Ca++-sensitive microelectrodes depends on the cell type and the magnitude and time constant of the event under study (Borle (1990) Environ Health Perspect 84:45-56). As an exemplary method of Ca++ detection, cells could be loaded with the Ca++ sensitive fluorescent dye fura-2 or indo-1, using standard methods, and any change in Ca++ measured using a fluorometer.

As certain embodiments described above suggest, in addition to directly measuring second messenger production, the signal transduction activity of a receptor or ion channel pathway can be measured by detection of a transcription product, e.g., by detecting receptor/channel-mediated transcriptional activation (or repression) of a gene(s). Detection of the transcription product includes detecting the gene transcript, detecting the product directly (e.g., by immunoassay) or detecting an activity of the protein (e.g., such as an enzymatic activity or chromogenic/fluorogenic activity); each of which is generally referred to herein as a means for detecting expression of the indicator gene. The indicator gene may be an unmodified endogenous gene of the host cell, a modified endogenous gene, or a part of a completely heterologous construct, e.g., as part of a reporter gene construct.

In one embodiment, the indicator gene is an unmodified endogenous gene. In certain instances, it may be desirable to increase the level of transcriptional activation of the endogenous indicator gene by the signal pathway in order to, for example, improve the signal-to-noise of the test system, or to adjust the level of response to a level suitable for a particular detection technique. In one embodiment, the transcriptional activation ability of the signal pathway can be amplified by the overexpression of one or more of the proteins involved in the intracellular signal cascade, particularly enzymes involved in the pathway. For example, increased expression of Jun kinases (JNKs) can potentiate the level of transcriptional activation by a signal in an MEK/MEKK pathway. This approach can, of course, also be used to potentiate the level of transcription of a heterologous reporter gene as well.

In other embodiments, the sensitivity of an endogenous indicator gene can be enhanced by manipulating the promoter sequence at the natural locus for the indicator gene. Such manipulation may range from point mutations to the endogenous regulatory elements to gross replacement of all or substantial portions of the regulatory elements. In general, manipulation of the genomic sequence for the indicator gene can be carried out using techniques known in the art, including homologous recombination.

In another exemplary embodiment, the promoter (or other transcriptional regulatory sequences) of the endogenous gene can be “switched out” with a heterologous promoter sequence, e.g., to form a chimeric gene at the indicator gene locus. Again, using such techniques as homologous recombination, the regulatory sequence can be so altered at the genomic locus of the indicator gene.

In still another embodiment, a heterologous reporter gene construct can be used to provide the function of an indicator gene. Reporter gene constructs are prepared by operatively linking a reporter gene with at least one transcriptional regulatory element. If only one transcriptional regulatory element is included it must be a regulatable promoter, At least one the selected transcriptional regulatory elements must be indirectly or directly regulated by the activity of the selected cell-surface receptor whereby activity of the receptor can be monitored via transcription of the reporter genes.

Many reporter genes and transcriptional regulatory elements are known to those of skill in the art and others may be identified or synthesized by methods known to those of skill in the art.

g. Exemplary Screening and Selection Assays: Reporter Genes

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

Transcriptional control elements for use in the reporter gene constructs, or for modifying the genomic locus of an indicator gene include, but are not limited to, promoters, enhancers, and repressor and activator binding sites. Suitable transcriptional regulatory elements may be derived from the transcriptional regulatory regions of genes whose expression is rapidly induced, generally within minutes, of contact between the cell surface protein and the effector protein that modulates the activity of the cell surface protein. Examples of such genes include, but are not limited to, the immediate early genes (see, Sheng et al. (1990) Neuron 4: 477-485), such as c-fos. Immediate early genes are genes that are rapidly induced upon binding of a ligand to a cell surface protein. The transcriptional control elements that are preferred for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

Other promoters and transcriptional control elements, in addition to those described above, include the vasoactive intestinal peptide (VIP) gene promoter (cAMP responsive; Fink et al. (1988), Proc. Natl. Acad. Sci. 85:6662-6666); the somatostatin gene promoter (cAMP responsive; Montminy et al. (1986), Proc. Natl. Acad. Sci. 8.3:6682-6686); the proenkephalin promoter (responsive to cAMP, nicotinic agonists, and phorbol esters; Comb et al. (1986), Nature 323:353-356); the phosphoenolpyruvate carboxy-kinase gene promoter (cAMP responsive; Short et al. (1986), J. Biol. Chem. 261:9721-9726); the NGFI-A gene promoter (responsive to NGF, cAMP, and serum; Changelian et al. (1989). Proc. Natl. Acad. Sci. 86:377-381); and others that may be known to or prepared by those of skill in the art.

In the case of receptors which modulate cyclic AMP, a transcriptional based readout can be constructed using the cyclic AMP response element binding protein, CREB, which is a transcription factor whose activity is regulated by phosphorylation at a particular serine (S133). When this serine residue is phosphorylated, CREB binds to a recognition sequence known as a CRE (cAMP Responsive Element) found to the 5′ of promotors known to be responsive to elevated cAMP levels. Upon binding of phosphorylated CREB to a CRE, transcription from this promoter is increased.

Phosphorylation of CREB is seen in response to both increased cAMP levels and increased intracellular Ca levels. Increased cAMP levels result in activation of PKA, which in turn phosphorylates CREB and leads to binding to CRE and transcriptional activation. Increased intracellular calcium levels results in activation of calcium/calmodulin responsive kinase IV (CaM kinase IV). Phosphorylation of CREB by CaM kinase IV is effectively the same as phosphorylation of CREB by PKA, and results in transcriptional activation of CRE containing promotors. Activation of extracellular signal-related protein kinase (ERK) and Rsk2 by also leads to the phosphorylation and transactivation of CREB. Impey et al. (1998) Neuron 21:869-883.

Therefore, a transcriptional-based readout can be constructed in cells containing a reporter gene whose expression is driven by a basal promoter containing one or more CRE. Changes in the intracellular concentration of Ca++ (a result of alterations in the activity of the receptor upon engagement with a ligand) will result in changes in the level of expression of the reporter gene if: a) CREB is also co-expressed in the cell, and b) either the endogenous yeast CaM kinase will phosphorylate CREB in response to increases in calcium or if an exogenously expressed CaM kinase IV is present in the same cell. In other words, stimulation of PLC activity will result in phosphorylation of CREB and increased transcription from the CRE-construct, while inhibition of PLC activity will result in decreased transcription from the CRE-responsive construct.

As described in Bonni et al. (1993) Science 262:1575-1579, the observation that CNTF treatment of SK-N-MC cells leads to the enhanced interaction of STAT/p91 and STAT related proteins with specific DNA sequences suggested that these proteins might be key regulators of changes in gene expression that are triggered by CNTF. Consistent with this possibility is the finding that DNA sequence elements similar to the consensus DNA sequence required for STAT/p91 binding are present upstream of a number of genes previously found to be induced by CNTF (e.g., Human c-fos, Mouse c-fos, Mouse tis11, Rat junB, Rat SOD-1, and CNTF). Those authors demonstrated the ability of STAT/p91 binding sites to confer CNTF responsiveness to a non-responsive reporter gene. Accordingly, a reporter construct for use in the present invention for detecting signal transduction through STAT proteins, such as from cytokine receptors, can be generated by using −71 to +109 of the mouse c-fos gene fused to the bacterial chloramphenicol acetyltransferase gene (−71 fosCAT) or other detectable marker gene. Induction by a cytokine receptor induces the tyrosine phosphorylation of STAT and STAT-related proteins, with subsequent translocation and binding of these proteins to the STAT-RE. This then leads to activation of transcription of genes containing this DNA element within their promoters.

In preferred embodiments, the reporter gene is a gene whose expression causes a phenotypic change which is screenable or selectable. If the change is selectable, the phenotypic change creates a difference in the growth or survival rate between cells which express the reporter gene and those which do not. If the change is screenable, the phenotype change creates a difference in some detectable characteristic of the cells, by which the cells which express the marker may be distinguished from those which do not. Selection is preferable to screening in that it can provide a means for amplifying from the cell culture those cells which express a test polypeptide which is a receptor effector.

The marker gene is coupled to the receptor signaling pathway so that expression of the marker gene is dependent on activation of the receptor. This coupling may be achieved by operably linking the marker gene to a receptor-responsive promoter. The term “receptor-responsive promoter” indicates a promoter which is regulated by some product of the target receptor's signal transduction pathway.

Alternatively, the promoter may be one which is repressed by the receptor pathway, thereby preventing expression of a product which is deleterious to the cell. With a receptor repressed promoter, one screens for agonists by linking the promoter to a deleterious gene, and for antagonists, by linking it to a beneficial gene. Repression may be achieved by operably linking a receptor-induced promoter to a gene encoding mRNA which is antisense to at least a portion of the mRNA encoded by the marker gene (whether in the coding or flanking regions), so as to inhibit translation of that mRNA. Repression may also be obtained by linking a receptor-induced promoter to a gene encoding a DNA binding repressor protein, and incorporating a suitable operator site into the promoter or other suitable region of the marker gene.

h. Exemplary Emodiments: Host Cells

Suitable host cells for generating the subject assay include prokaryotes, yeast, or higher eukaryotic cells, especially mammalian cells. Prokaryotes include gram negative or gram positive organisms. Examples of suitable mammalian host cell lines include the COS-7 line of monkey kidney cells (ATCC CRL 1651) (Gluzman (1981) Cell 23:175) CV-1 cells (ATCC CCL 70), L cells, C127, 3T3, Chinese hamster ovary (CHO), HeLa and BHK cell lines. It will be understood that to achieve selection or screening, the host cell must have an appropriate phenotype.

If yeast cells are used, the yeast may be of any species which are cultivable and in which an exogenous receptor can be made to engage the appropriate signal transduction machinery of the host cell. Suitable species include Kluyverei lactis, Schizosaccharomyces pombe, and Ustilaqo maydis; Saccharomyces cerevisiae is preferred. Other yeast which can be used in practicing the present invention are Neurospora crassa, Aspergillus niger, Aspergillus nidulans, Pichia pastoris, Candida tropicalis, and Hansenula polymorpha. The term “yeast”, as used herein, includes not only yeast in a strictly taxonomic sense, i.e., unicellular organisms, but also yeast-like multicellular fungi or filamentous fungi.

The choice of appropriate host cell will also be influenced by the choice of detection signal. For instance, reporter constructs, as described below, can provide a selectable or screenable trait upon transcriptional activation (or inactivation) in response to a signal transduction pathway coupled to LTM protein of interest. The reporter gene may be an unmodified gene already in the host cell pathway, such as the genes responsible for growth arrest in yeast. It may be a host cell gene that has been operably linked to a “receptor-responsive” promoter. Alternatively, it may be a heterologous gene (e.g., a “reporter gene construct”) that has been so linked. Suitable genes and promoters are discussed below. In other embodiments, second messenger generation can be measured directly in the detection step, such as mobilization of intracellular calcium or phospholipid metabolism are quantitated. In yet other embodiments indicator genes can be used to detect receptor-mediated signaling.

E. Use of Animal Models for Validating Drug Leads

Compounds which are identified as active in the above-described assays can be further assessed for in vivo efficacy in regulating LTM in a variety of animal systems.

In one embodment, such compounds can be tested in an animal model for studying fornix-mediated memory consolidation, e.g., an animal which as been prepared by introducing lesions to the formix structure. As we have previously described (Taubenfeld et al. (1999) Nat Neurosci 2:309), animals with fornix lesions learn the inhibitory avoidance and display memory at control levels for up to 6 hours, however, by 24 hours they exhibit amnesia.

In general, the subject method utilizes an animal which has been manipulated to create at least partial disruption of fornix-mediated signalling to the hippocampus, the disruption affecting memory consolidation and learned behavior in the animal. The animal is conditioned with a learning or memory regimen which results in learned behavior in the mammal in the absence of the fornix lesion. Test agents are administered to the animal in order assess their effects on memory consolidation. An increase in learned behavior, relative to the absence of the test agent, indicates that the test agent enhances memory consolidation.

In the methods of the present invention, retention of the learned behavior can be determined, for example, after at least about 12-24 hours, 14-22 hours, 16-20 hours and or 18-19 hours after completion of the learning phase to determine whether the agent promotes memory consolidation. In a particular embodiment, retention of the learned behavior can be determined 24 hours after completion of the learning phase.

In the methods of the present invention, the lesion mammal can have a lesion of the fornix or a related brain structure that disrupts memory consolidation (e.g., perirhinal cortex, amygdala, medial septal nucleus, locus coeruleus, hippocampus, mammallary bodies). Lesions in the mammal can be produced by mechanical or chemical disruption. For example, the fornix lesion can be caused by surgical ablation, electrolytic, neurotoxic and other chemical ablation techniques, or reversible inactivation such as by injection of an anesthetic, e.g., tetrodooxin or lidocaine, to temporarily arrest activity in the fornix.

To further illustrate, fimbrio-fornix (rodents) and fornix (primates) lesions can be created by stereotatic ablation. In particular, neurons of the fornix structure are axotomized, e.g., by transection or aspiration (suction) ablation. A complete transection of the fornix disrupts cholinergic and GABAergic function and electrical activity, and induces morphological reorganization in the hippocampal formation. In general, the fornix transection utilized in the subject method will not disconnect the parahippocampal region from the neocortex. In those embodiments, the fornix transection will not disrupt functions that can be carried out by the parahippocampal region independent of processing by the hippocampal formation, and hence would not be expected to produce the full-blown amnesia seen following more complete hippocampal system damage.

In one embodiment, the animal can be a rat. Briefly, the animals are anesthetized, e.g., with intraperitoneal injections of a ketamine-xylazine mixture and positioned in a Kopf stereotoxic instrument. A sagittal incision is made in the scalp and a craniotomy is performed extending 2.0 mm posterior and 3.0 mm lateral from Bregma. An aspirative device, e.g., with a 20 gauge tip, is mounted to a stereotaxic frame (Kopf Instruments) and fimbria-fornix is aspirated by placing the suction tip at the correct sterotaxic location in the animals brain. Unilateral aspirative lesions are made by suction through the cingulate cortex, completely transecting the fimbria fornix unilaterally, and (optionally) removing the dorsal tip of the hippocampus as well as the overlying cingulate cortex to inflict a partial denervation on the hippocampus target. See also, Gage et al., (1983) Brain Res. 268:27 and Gage et al. (1986) Neuroscience 19:241.

In another exemplary embodiment, the animal can be a monkey. The animals can be anesthetized, e.g., with isoflurane (1.5-2.0%). Following pretreatment with mannitol (0.25 g/kg, iv), unilateral transections of the left fornix an be performed, such as described by Kordower et al. (1990) J. Comp. Neurol., 298:443. Briefly, a surgical drill is used to create a parasagittal bone flap which exposes the frontal superior sagittal sinus. The dura is retracted and a self-retaining retractor is used to expose the interhemispheric fissure. The corpus callosum is longitudinally incised. At the level of the foramen of Monro, the fornix is easily visualized as a discrete 2-3 mm wide white fiber bundle. The fornix can be initially transected using a ball dissector. The cut ends of the fornix can then be suctioned to ensure completeness of the lesion.

In still other illustrative embodiments, the fornix lesion can be created by excitotoxically, or by other chemical means, inhibiting or ablating fornix neurons, or the cells of the hippocampus which are innervated by fornix neurons. In certain preferred embodiments, the fornix lesion is generated by selective disruption of particular neuronal types, such as fornix cholinergic, GABAergic and/or serotonergic neurons, and in certain embodiments, particular morphological subtypes within such neuron types. For instance, selective ablation of serotonergic neurons can be accomplished by treatment of the fornix structure with methamphetamines, such as d-methamphetamine (d-MA), methylenedioxyamphetamine (MDA) and methylenedioxymethamphetamine (MDMA), and 5,7-dihydroxytryptamine (5,7-DHT). The afferant fornix signals to the hippocampus due to cholinergic neurons can be ablated by atropine blockade. Another means for ablation of the cholinergic neurons is the use of 192IgG-saporin (192IgG-sap), e.g., intraventricularly injection into the fornix and hippocampus. In other embodiments, the agents such as 6-OHDA and ibotenic acid can be used to selectively destroy fornix dopamine neurons as part of the ablative regimen. Other exemplary agents which may used to create fornix lesions include N-methyl-D-aspartate (NMDA), quinolinic acid, and methylazoxymethanol.

There are a variety of test for cognitive function, especially leaming and memory testing, which can be carried our using the lesioned and normal animals. Learning and/or memory tests include, for example, inhibitory avoidance, contextual fear conditioning, visual delay non-match to sample, spatial delay non-match to sample, visual discrimination, Barnes circular maze, Morris water maze and Radial arm maze tests.

An exemplary inhibitory avoidance test utilizes an apparatus that consists of a lit chamber that can be separated from a dark chamber by a sliding door. At training, the animal is placed in the lit chamber for some period of time, then the door is opened, the animal moves to the dark chamber after a short delay-the latency, that is recorded. Upon entry into the dark chamber, the door is shut closed and a footshock is delivered. Retention of the experience is determined after various time intervals, e.g., 24 or 48 hours, by repeating the test and recording the latency. The protocol is one of many variants of the inhibitory avoidance procedures (for review, see Rush (1988) Behav Neural Biol 50:255).

An exemplary maze testing embodiment is the water maze working memory test. In general, the method utilizes an apparatus which consists of a circular water tank. The water in the tank is made cloudy by the addition of milk powder. A clear plexiglass platform, supported by a movable stand rest on the bottom of the tank, is submerged just below the water surface. Normally a swimming rat cannot perceive the location of the platform but it may recall it from a previous experience and training, unless it suffers from some memory impairment. The time taken to locate the platform is measured and referred to as the latency. During the experiment, all orientational cues such as ceiling lights etc. remain unchanged. Longer latencies are generally observed with rats with some impairment to their memory.

Another memory test includes the eyeblink conditioning test, which involves the administration of white noise or steady tone that preceeds a mild air puff which stimulates the subject's eyeblink.

Still another memory test which can be used is fear conditioning, e.g., either “cued” and “contextual” fear conditioning. In one embodiment, a freeze monitor administers a sequence of stimuli (sounds, shock) and then records a series of latencies measuring the recovery from shock induced freezing of the animal.

Another memory test for the lesioned animals is a holeboard test, which utilizes a rotating holeboard apparatus containing (four) open holes arranged in a 4-corner configuration in the floor of the test enclosure. A mouse is trained to poke its head into a hole and retrieve a food reward from a “baited” hole which contains a reward on every trial. There is a food reward (e.g. Froot Loop) in every exposed hole which is made inaccessible by being placed under a screen. The screen allows the odor of the reward to emanate from the hole, but does not allow access to the reinforcer. When an individual hole is baited, a small piece of Froot Loop is placed on top of the screen, where it is accessible. The entire apparatus rests on a turntable so that it may be rotated easily to eliminate reliance on proximal (e.g. olfactory) cues. A start tube is placed in the center of the apparatus. The subject is released from the tube and allowed to explore for the baited (“correct”) hole.

F. Pharmaceutical Preparations of Identified Agents

After identifying certain test compounds in the subject assay, e.g., as potentiators or inhibitors of memory consolidation, the practioner of the subject assay will continue to test the efficacy and specificity of the selected compounds both in vitro and in vivo. Whether for subsequent in vivo testing, or for administration to an animal as an approved drug, agents identified in the subject assay can be formulated in pharmaceutically acceptable excipients for in vivo administration to an animal, preferably a human.

The compounds selected in the subject assay, or a pharmaceutically acceptable salt thereof, may accordingly be formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the compound, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”. Based on the above, such pharmaceutical formulations include, although not exclusively, solutions or freeze-dried powders of the compound in association with one or more pharmaceutically acceptable vehicles or diluents, and contained in buffered media at a suitable pH and isosmotic with physiological fluids. In preferred embodiment, the compound can be disposed in a sterile preparation for topical and/or systemic administration. In the case of freeze-dried preparations, supporting excipients such as, but not exclusively, mannitol or glycine may be used and appropriate buffered solutions of the desired volume will be provided so as to obtain adequate isotonic buffered solutions of the desired pH. Similar solutions may also be used for the pharmaceutical compositions of compounds in isotonic solutions of the desired volume and include, but not exclusively, the use of buffered saline solutions with phosphate or citrate at suitable concentrations so as to obtain at all times isotonic pharmaceutical preparations of the desired pH, (for example, neutral pH).

In certain embodiment, the pharmaceutical of the present invention is a gene delivery system for gene therapy with a therapeutic LTM gene. Such gene therapy systems can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) Proc. Natl. Acad. Sci. USA 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

F. Methods of Treatment

In various embodiments, the present invention contemplates modes of treatment and prophylaxis which utilitize one or more of the subject LTM genes (e.g., by gene therapy) or antisense constructs thereto, the LTM proteins (e.g., for protein therapy) or peptidomimetics thereof, or compounds identified in the subject drug screening assays. These agents may be useful for altering (increasing or decreasing) the occurrence of learning and/or memory defects in an organism, and thus, altering the learning ability and/or memory capacity of the organism. In other embodiments, the preparations of the present invention can be used simply to enhance normal memory function.

Memory disorders which can be treated according to the present invention may have a number of origins: a functional mechanism (anxiety, depression), physiological aging (age-associated memory impairment), drugs, or anatomical lesions (dementia). Indications for which such preparations may be useful include learning disabilities, memory impairment, e.g., due to toxicant exposure, brain injury, age, schizophrenia, epilepsy, mental retardation in children and senile dementia, including Alzheimer's disease.

In certain embodiments, the invention contemplates the treatment of amnesia. Amnesias are described as specific defects in declarative memory. Faithful encoding of memory requires a registration, rehearsal, and retention of information. The first two elements appear to involve the hippocampus and medial temporal lobe structures. The retention or storage appears to involve the heteromodal association areas. Amnesia can be experienced as a loss of stored memory or an inability to form new memories. The loss of stored memories is known as retrograde amnesia. The inability to form new memories is known as anterograde amnesia.

Complaints of memory problems are common. Poor concentration, poor arousal and poor attention all may disrupt the memory process to a degree. The subjective complaint of memory problems therefore must be distinguished from true amnesias. This is usually done at the bedside in a more gross evaluation and through specific neuropsychological test. Defects in visual and verbal memory can be separated through such test. In amnesias there is by definition a preservation of other mental capacities such as logic. The neurobiologic theory of memory described above would predict that amnesias would have relatively few pathobiologic variations. Clinically the problem of amnesias often presents as a result of a sudden illness in an otherwise healthy person.

Exemplary forms of amnesias which may be treated by the subject method include, amensias of short duration, alcoholic blackouts, Wemicke-Korsakoff's (early), partial complex seizures, transient global amnesia, those which are medication related, such as triazolam (Halcion), basilar artery migraines. The subject method may also be used to treat amensias of longer duration, such as post concussive or as the result of Herpes simplex encephalitis.

(i) Effective Dose

Toxicity and therapeutic efficacy of compounds to be used in the treatment methods of the present invention can be determined by standard pharmaceutical procedures in cell cultures or experimental animals, e.g., for determining The LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

G. Diagnostic and Prognostic Assays

The present method also provides a method for determining if a subject is at risk for a disorder characterized deterioration of memory consolidation. In preferred embodiments, the methods can be characterized as comprising detecting, in a sample of cells from the subject, the presence or absence of a genetic lesion characterized by at least one of (i) an alteration affecting the integrity of a gene encoding an LTM protein, or (ii) the mis-expression of the LTM gene. To illustrate, such genetic lesions can be detected by ascertaining the existence of at least one of (i) a deletion of one or more nucleotides from an LTM gene, (ii) an addition of one or more nuclectides to an LTM gene, (iii) a substitution of one or more nucleotides of an LTM gene, (iv) a gross chromosomal rearrangement of an LTM gene, (v) a gross alteration in the level of a messenger RNA transcript of an LTM gene, (vii) aberrant modification of an LTM gene, such as of the methylation pattern of the genomic DNA, (vii) the presence of a non-wild type splicing pattern of a messenger RNA transcript of an LTM gene, (viii) a non-wild type level of an LTM-protein, (ix) allelic loss of an LTM gene, and (x) inappropriate post-translational modification of an LTM-protein. As set out below, the present invention provides a large number of assay techniques for detecting lesions in an LTM gene, and importantly, provides the ability to discern between different molecular causes underlying a disorder.

In an exemplary embodiment, there is provided a nucleic acid composition comprising a (purified) oligonucleotide probe including a region of nucleotide sequence which is capable of hybridizing to a sense or antisense sequence of an LTM gene, such as represented by any of SEQ ID NOs: 1, 3, 5, 7, 9 or 11, or naturally occurring mutants thereof, or 5′ or 3′ flanking sequences or intronic sequences naturally associated with the subject LTM genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

In preferred embodiments, the method can be generally characterized as comprising detecting, in a sample of cells from the subject, the presence or absence of a genetic lesion characterized by an alteration affecting the integrity of an LTM gene. To illustrate, such genetic lesions can be detected by ascertaining the existence of at least one of (i) a deletion of one or more nucleotides from an LTM gene, (ii) an addition of one or more nucleotides to an LTM gene, (iii) a substitution of one or more nucleotides of an LTM gene, and (iv) the presence of a non-wild type splicing pattern of a messenger RNA transcript of an LTM gene. As set out below, the present invention provides a large number of assay techniques for detecting lesions in LTM genes.

In certain embodiments, detection of the lesion comprises utilizing the probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the LTM gene (see Abravaya et al. (1995) Nuc Acid Res 23:675-682). In a merely illustrative embodiment, the method includes the steps of (i) collecting a sample of cells from a patient, (ii) isolating nucleic acid (c .g., genomic, mRNA or both) from the cells of the sample, (iii) contacting the nucleic acid sample with one or more primers which specifically hybridize to an LTM gene under conditions such that hybridization and amplification of the LTM gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Another embodiment of the invention provides for a nucleic acid composition comprising a (purified) oligonucleotide probe including a region of nucleotide sequence which is capable of hybridizing to a sense or antisense sequence of an LTM gene, or naturally occurring mutants thereof, or 5′ or 3′ flanking sequences or intronic sequences naturally associated with the subject LTM genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels. Such oligonucleotide probes can be used for both predictive and therapeutic evaluation of allelic mutations which might be manifest in, for example, deterioration in memeory consolidation.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving memory or an LTM gene.

Antibodies directed against wild type or mutant LTM proteins, which are discussed, above, may also be used in disease diagnostics and prognostics. Such diagnostic methods, may be used to detect abnormalities in the level of LTM protein expression, or abnormalities in the structure and/or tissue, cellular, or subcellular location of LTM protein. Structural differences may include, for example, differences in the size, electronegativity, or antigenicity of the mutant LTM protein relative to the normal LTM protein. Protein from the tissue or cell type to be analyzed may easily be detected or isolated using techniques which are well known to one of skill in the art, including but not limited to western blot analysis. For a detailed explanation of methods for carrying out western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of LTM proteins. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the LTM protein, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-LTM protein specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in an LTM gene or gene product can be used to monitor the course of treatment or therapy.

H. Transgenic Animals

These systems may be used in a variety of applications. For example, the cell- and animal-based model systems may be used to further characterize the role of the subject LTM genes and proteins in memory. In addition, such assays may be utilized as part of screening strategies designed to identify compounds which are capable of ameliorating disease symptoms. Thus, the animal- and cell-based models may be used to identify drugs, pharmaceuticals, therapies and interventions which may be effective in treating disease.

One aspect of the present invention concerns the use of a transgenic animal which is comprised of cells (of that animal) which contain a transgene of the present invention and which preferably (though optionally) express an exogenous LTM protein in one or more cells in the animal. An LTM transgene can encode the wild-type form of the protein, or can encode homologs thereof including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of an LTM protein can be essential for many forms of lineage analysis and can additionally provide a means to assess the effects of, for example, lack of LTM expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2^{b} , H-2^{d} or H-2q haplotypes such as C57BL/6 or DBA/1. The line(s) used to practice this invention may themselves be transgenics, and/or may be knockouts (i.e., obtained from animals which have one or more genes partially or (completely suppressed).

In one embodiment, gene targeting, which is a method of using homologous recombination to modify an animal's genome, can be used to introduce changes into cultured embryonic stem cells. By targeting an LTM gene of interest in ES cells, these changes can be introduced into the germlines of animals to generate chimeras. The gene targeting procedure is accomplished by introducing into tissue culture cells a DNA targeting construct that includes a segment homologous to a target LTM locus, and which also includes an intended sequence modification to the LTM genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting an LTM gene function through the use of a targeting transgene construct designed to undergo homologous recombination with one or more LTM genomic sequences. The targeting constrict can be arranged so that, upon recombination with an element of an LTM gene, a positive selection marker is inserted into (or replaces) coding sequences of the targeted gene. The inserted sequence functionally disrupts the LTM gene, while also providing a positive selection trait. Exemplary LTM targeting constructs are described in more detail below.

The present invention is further illustrated by the following examples which should not be construed as limiting in any way. The contents of all cited references (including literature references, issued patents, published patent applications as cited throughout this application are hereby expressly incorporated by reference.

Exemplification

### EXAMPLE 1

Northern Blot Analysis Reveals That Several Immediate-early Genes are Induced Following IA Training and That This Training-related Gene Regulation is Sensitive to Fornix Lesions.

In the invertebrate Aplysia californica, it has been demonstrated that the gene expression changes which underlie memory formation begin with the induction of regulatory immediate-early genes (IEGs). Specifically, induction of ApC/EBP, a member of the CCAAT enhancer binding protein (C/EBP) transcription factor family, is essential for long-term synaptic plasticity that underlies memory formation in Aplysia (Alberini, 1994). Based on these data, we proposed that memory-inducing stimuli activate a gene cascade, where CREB regulates the expression of regulatory IEGs which, in turn, regulate the expression of more downstream target genes required for long-term memory (Alberini, 1994). This model accounts for why gene expression seems to be required only for an early and brief time window. The essential gene expression appears to be brief because it corresponds to the critical time necessary for the expression of regulatory IEGs, which would be the rate-limiting step of the molecular cascade of events leading to long-term memory.

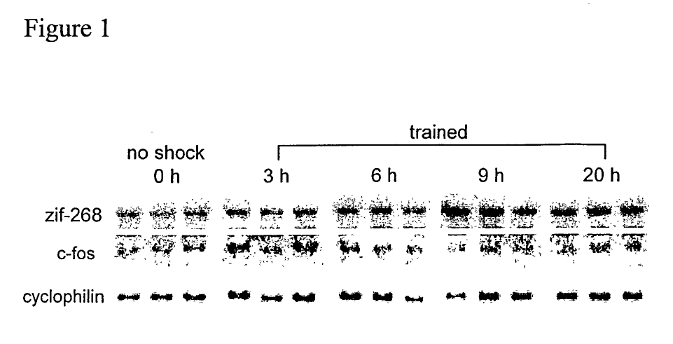

In mammals, changes in mRNA and protein levels of IEGs such as c-fos and zif268 have been widely investigated in memory and models of synaptic plasticity. Although still controversial (Campeau, 1991), particularly due to the lack of precise controls or quantitative measurements (the studies have been generally based on immunohistochemical analysis), both c-fos and zif268 have been reported to be up-regulated in several brain areas following different kinds of learning (rev. in Dragunow, 1996). Therefore, in our initial pilot experiments, we performed a Northern blot analysis of changes in hippocampal zif268 and c-fos mRNA levels following IA training, focusing on the temporal window suggested by induction of PCREB; i.e., 3, 6, 9, and 20 hrs after training. Three animals per timepoint were investigated, and the hybridizations were normalized using cyclophilin gene as a control probe. The same membrane was sequentially hybridized with all the probes. As shown in FIG. 1, we found that zif268 was induced in all the trained animals at 9 and 20 hr after training. On the contrary, c-fos did not show any evident change throughout the timecourse. Thus, the IEG response to IA training are selective with respect to the genes activated as well as the time following training.

FIG. 1. Time-course Northern blot analysis of zif268 and c-fos following IA training. Increase in zif268, but not c-fos mRNA is evident in all animals at 9 and 20 hr after training. These results showed that zif268 is induced in the hippocampus by IA. They also reveal when the induction of an IEG is detectable in the hippocampus following IA, namely at 9 and 20 hrs. On the basis of these data, as described below, we decided to perform our first array hybridization analysis using the RNA obtained from animals 9 hr after training.

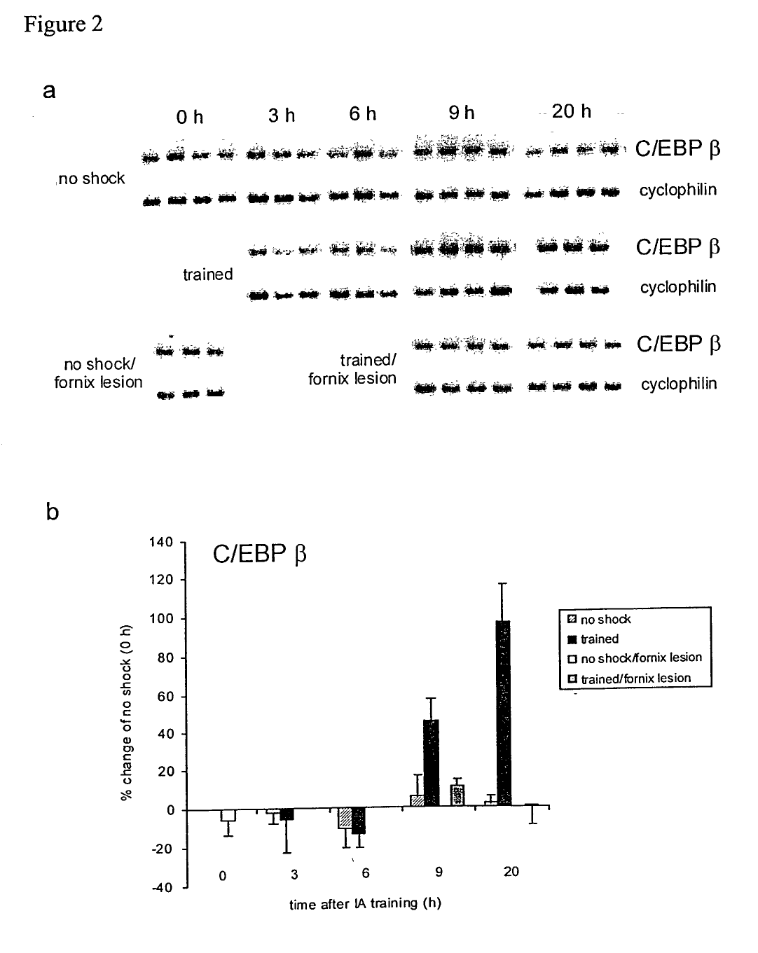

Encouraged by these results with zif268, we subsequently hybridized the same blot using probes directed at additional transcription factors, including C/EBPβ a member of the CCAAT enhancer binding protein family. We found that C/EBPβ was induced following IA training during a time window similar to that of zif268 (FIG. 2). This finding is extremely compelling, since it suggests the possibility that like CREB, members of the C/EBP family, might be evolutionarily conserved components of the gene cascade activated during memory formation.

To define whether the increase in C/EBPβ expression is related to IA memory rather than exploring a new environment, the IA trained animals were compared at each timepoint to paired-control animals that walked through the inhibitory avoidance apparatus without receiving a footshock in the dark chamber. Moreover, the level of hippocampal C/EBPβ mRNA did not change in animals that received only the shock without the contextual learning and were sacrificed 9 and 20 hr later (not shown). This is not surprising since both learning (Fanselow, 1990) and an increase in PCREB (Taubenfeld et al. 1999) do not occur in animals that received the shock only. Therefore, C/EBPβ seems to be selectively induced by IA training.

We then began to address the question of whether the C/EBPβ response that we found to be associated with memory is sensitive to fornix lesions. As shown in FIG. 2, animals with lesions failed to show any change in the expression of C/EBPβ after IA training.

FIG. 2. Time course Northern blot analysis of C/EBPβ and cyclophilin (control) mRNA following IA training in hippocampi of unoperated and fornix-lesion rats. Importantly, the level of hippocampal C/EBPβ in animals with fornix lesions does not differ from that of unoperated animals (compare 0 no shock and 0 no shoch fornix lesion in FIG. 2). This finding clearly demonstrates that the fornix is specifically required for this gene response to IA training, and considerably strengthens the hypothesis that the changes we have observed are selectively related to memory consolidation.

### EXAMPLE 2

Array Hybridization Reveals New Gene Responses to IA Training.

We believe that our model system lends itself particularly well to searching for genes differentially expressed in long-term memory. We have determined where and when to carry out the differential analysis in animals that have the ability to learn vs. those with memory impairment. We also know that learning induces a hippocampal gene response that is sensitive to fornix lesions.

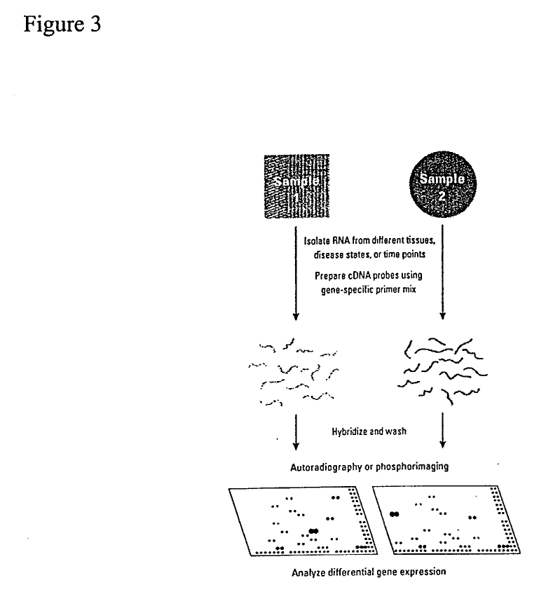

The possibility of identifying differentially expressed genes is one of the most powerful approaches for understanding the gene pattern linked to a specific function. A multitude of techniques have become available in recent years to isolate differentially expressed genes and some have been successfully used in isolating genes involved in long-term memory (Cavallaro, 1997). The most advanced generation of these techniques is the hybridization of DNA arrays. The arrays consist of supports (nylon or glass) on which cDNA fragments have been immobilized systematically. A schematic representation of how this technique works is shown in FIG. 3.

FIG. 3. Broad scale expression profiling with cDNA expression arrays. Side by side hybridizations with cDNA probes prepared from two different RNA populations allow the simultaneous comparison of the expression levels of all the cDNAs on the array. (from Clontech user manual). Arrays can contain, in principle, all the cloned DNA sequences. We begun our analysis using a relatively small array, the AtlasTM rat cDNA expression array from Clontech (Palo Alto, Calif.), which was the most complete commercially available array at that time. This array contained 588 genes isolated from rat brain and coding for a variety of molecules involved in the pathways that regulate brain function, such as signaling molecules, receptors, signal transduction proteins, extracellular proteins, structural molecules, molecules involved in synaptic transmission, and molecules involved in neural pathologies, including Alzheimer disease. The DNAs were fixed on a positively charged nylon membrane.

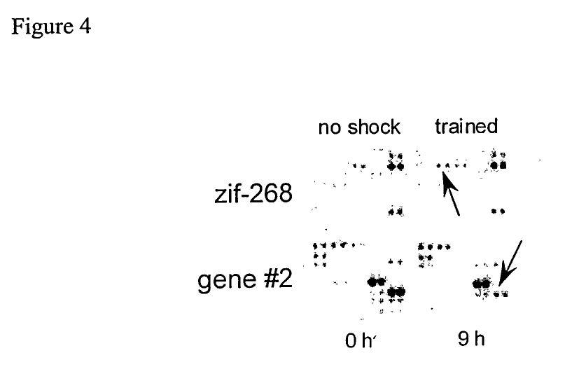

Since it is possible to detect changes in mRNA expression at 9 hr after IA training compared to controls, we began the search for differential gene expression in memory using array hybridization and compared animals that walked through the apparatus without receiving a shock (0 no shock) and were immediately sacrificed to animals that underwent IA training and were sacrificed 9 hr later. Hippocampi from 4 control (0 no shock) and 4 trained animals were dissected, and their RNAs were isolated and pooled. The pooled RNAs from control and trained animals were used to hybridize two identical membranes. The array hybridizations appeared very clean; only a few spots out of 588 carried on the membranes showed changes in their hybridization signal. Importantly, these spots that showed changes in intensity were surrounded by others that had very comparable hybridization signals. In all, 9 hr after IA training, we found changes in the concentration of approximately 15 transcripts, eight of which have been analyzed and confirmed by Northern analysis. FIG. 4 shows two examples of our results using the arrays. (To protect the confidentiality of the results, the names of some genes are not revealed. They are identified with numbers. However if this presents an obstacle for considering the present application, the identity of the genes will be disclosed.) As described in FIG. 4, we found an increase in a spot that corresponded to zif268. This confirmed that the array analysis is sensitive enough to detect changes we have observed with Northern blot hybridization. Another spot, here named gene #2 appeared to be clearly decreased.

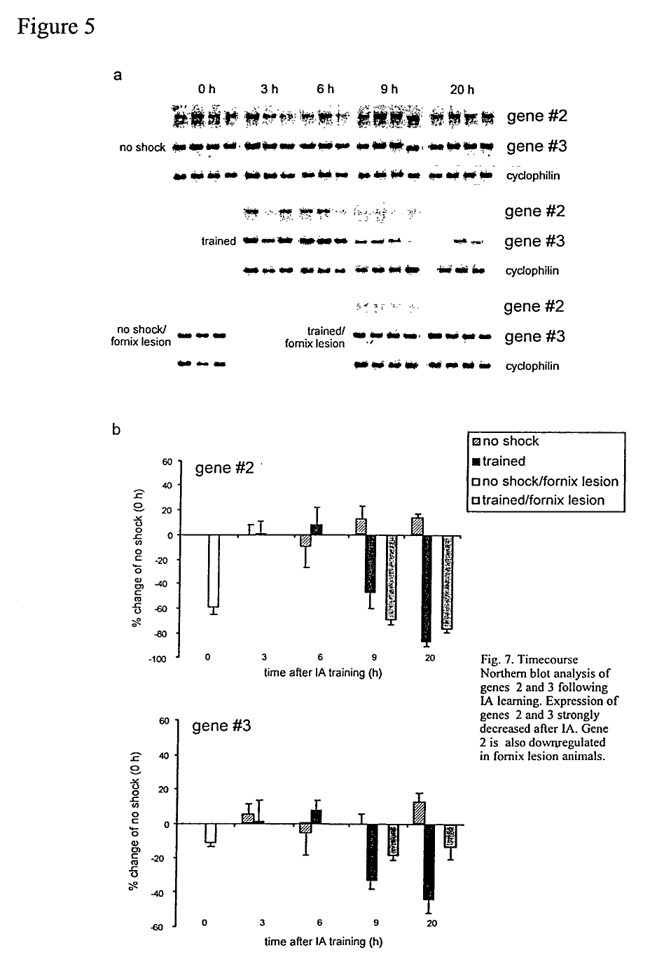

FIG. 4. Examples of changes in gene array hybridizations reflecting differential expression of mRNAs following IA training. Hippocampi of control rats are compared to hippocampi of rats trained and sacrificed 9 hr later. Note that on these arrays each sequence is spotted in duplicate. The array hybridization is a powerful method of screening. However, to prove that the transcripts identified with this technique indeed change their expression following IA learning, a Northern blot hybridization analysis of each identified sequence was necessary. The Northern blot, and not the array hybridization, ultimately defines whether a gene changes its expression after learning, therefore all the necessary controls were carried out with the Northern blot hybridization and not with array hybridization. The same membrane carrying the time-course analysis described above in FIG. 4, used to analyze C/EBPβ, was subsequently rehybridized to analyze the expression of gene #2 and of another gene that also appeared to decrease in the array hybridizations and that we called gene #3. As shown in FIG. 5, the Northern blot test confirmed that the levels of both transcripts #2 and 3 were strongly decreased at 9 and 20 hr post-training. No changes were observed at 3 and 6 hr after IA, and no significant changes were detected in paired control animals at any timepoint.

These results show that the regulation of genes #2 and #3 is selectively associated with IA training. As with C/EBPβ, the regulation of gene #2 that occurred with learning was abolished in fornix-lesioned animals, although the basal expression of the gene was not affected by lesioning. These data confirmed the IA memory formation is accompanied by regulation of gene expression that is induced by inputs passing through the fornix. Gene #3, on the other hand, appeared to be down-regulated with IA memory as well as in all animals with fornix lesions. From these data we conclude that fornix lesions produce changes in the regulation of certain genes within the hippocampus. This result is intriguing and suggests that the fornix may contribute to memory formation by modulating either constitutive or IA-induced gene expression.

Taken together, these data demonstrate that we are able to detect and analyze changes in gene expression after IA training. We have confirmed that some of these changes do not occur in the hippocampi of animals with lesions of the fornix which have impaired memory consolidation. We believe that these preliminary data show convincingly that our IA model is suitable for the analysis of gene expression changes in long-term memory.

We recently began to establish hybridization conditions of more complete arrays that have very recently become available from Research Genetics. These arrays contain 5,000 rat transcripts. Our goal is to carry out a systematic analysis of the rat cloned genes and identify which change their expression following training. To screen for genes whose expression change at different times after learning, as described in the Research Plan section, we plan to analyze several time windows after training and follow up with Northern blot analyses. Moreover, to define whether the gene cascade activated in IA is a general molecular mechanism of memory, we will analyze the expression of the identified genes in other forms of memory, including contextual fear conditioning and Morris water maze.

### EXAMPLE 3

The Delayed and Prolonged Gene Response During Memory Consolidation Makes Feasible Detection of Changes After the Morris Water Maze.

One hopes that the genes involved in long-term memory consolidation are conserved, not only evolutionarily, but also across different types of memory that utilize the same neural structures. We chose IA training originally because it could be used to precisely define the time-course of gene activation. Our survey of PCREB changes in the brain led us to focus our attention on the hippocampus. The hippocampus is also essential for the expression of memory probed using the Morris water maze (Riedel, 1999). Water maze training was not our first choice for investigating the gene response during memory consolidation because it typically takes many trials delivered over several days. However, the data from IA training suggest that fairly prolonged gene responses can lag behind the initial training period for up to 24 hrs. We have therefore trained rats in the water maze to investigate how much memory is demonstrated 24 hrs after a single day of training.

The Morris water maze is a very reliable and widely used assay of hippocampal function. Rats with damage to any part of the hippocampal system including the hippocampal formation itself, the entorhinal cortex and fornix show severe deficits on this task (Hannenson, 1998; Sutherland, 1989). The water maze apparatus consists of a large, circular pool filled with water made opaque by the addition of white tempura paint. An escape platform is hidden just below the surface of the water, and the rat is required to swim to the hidden platform from different starting points around the edge of the pool. Normal rats can acquire this task quickly by using and remembering configurations of extramaze cues to navigate directly to the platform. Learning is reflected in shorter latencies to escape and decreases in the length of the path that the rat swims in order to reach the platform.

In the following experiment, control rats (n=6) were trained on the water maze in order to determine how much information can be learned and remembered over a relatively brief training period. Each rat received eight trials a day for four consecutive days. As can be seen from FIG. 8 control rats learned to swim directly to the platform very quickly, reaching asymptotic performance after three days of training. A substantial amount of learning was seen after just one day of training, as mean escape latency decreased from 39.88 seconds on day one to 16.81 seconds on day 2 (see also FIG. 8). These results indicate that a reasonably robust spatial memory is formed after one day of training on the water maze. This task, therefore, may be included in our analysis of gene expression changes in long-term memory.

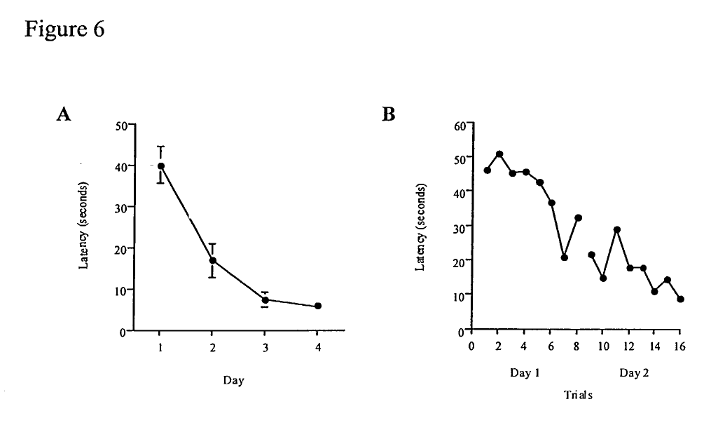

FIG. 6. Panel A. Mean escape latency for rats trained on the water maze. Rats received eight trials a day, for four consecutive days. Panel B. Mean escape latency on each of the eight trials of day one (trials 1-8) and day two (trials 9-16).

### EXAMPLE 4

Changes in Gene Expression Detected by Hybridizations of Array.

In this project we will identify which genes, among approximately 5,000 transcripts including genes and expressed sequence tag (ESTs), are regulated in the hippocampus during IA memory formation. We will compare parallel hybridizations of identical arrays with cDNA probes obtained from hippocampal RNA of untrained and trained animals. The transcripts that will show a significant change in expression in the trained condition ws the untrained will be further analyzed by Northern blot, following the sequence of steps described above in 1 to 4.

The array hybridization screening for genes differentially expressed following IA training will be carried on arrays purchased from Research Genetics (Huntsville, Ala.). This company has recently released the most complete rat DNA array commercially available, the rat GeneFilters microarrays. These arrays contain over 5,000 spots, representing approximately 1,700 named rat genes as well as many rat ESTs that are considered similar to named genes in other organisms. Each spot on the membrane contains approximately 0.5 ng of insert DNA from a cDNA clone containing the 3′ end of a gene. The insert cDNA has been denatured and UV-cross-linked to the positively charged membrane. The manufacturer provides a detailed protocol that insures that the hybridizations are carried out under conditions where most probes (cDNA from the experimental mRNAs) are not saturating the spots. However, as described below we will set up several hybridization conditions that will provide the highest probability of success for identifying genes regulated in memory. These arrays (nylon membranes) are similar to those we purchased from Clontech and successfully used to identify several genes, as described in Preliminary Studies. However, they contain a much greater number of transcripts. Finally, Research Genetics also provides a software analysis tool that, when used in conjunction with Gene Filters, allows for the comparison of gene expression from images produced on a phosphor imaging system. This system allows for normalization across multiple experiments and has a built-in database to facilitate archiving of both raw images and fitted data. To maximize the probability of detecting changes in gene expression levels following IA training we will perform multiple array hybridizations.

We claim:

1. A method for modulating long term memory consolidation in an animal comprising treating an animal with an agent that modulates the activity of one or more of zif268, insulin-like growth factor, glutamate receptor 1 (GluR1), glutamate receptor 2 (GluR2), c/EBPβ and VGF.

2. A method for enhancing long term memory consolidation in an animal comprising treating an animal with an agent that modulates a signal transduction pathway of glutamate receptor 1 (GluR1) or glutamate receptor 2 (GluR2), which agent is a ligand for the GluR1 or GluR2 receptor.

3. A method for identifying an agent which modulates memory consolidation, comprising,
(i) providing a reaction system for detecting the activity of a product encoded by a gene selected from the group consisting of of zif268, insulin-like growth factor, glutamate receptor 1 (GluR1), glutamate receptor 2 (GluR2), c/EBPβ and VGF;
(ii) contacting said system with a test compound; and
(iii) determining if the test compound alters the activity of the gene product.

4. A method for identifying an agent which modulates memory consolidation, comprising,
(i) providing a reaction system for detecting the level of expression of a gene selected from the group consisting of of zif 268, insulin-like growth factor, glutamate receptor 1 (GluR1), glutamate receptor 2 (GluR2), c/EBPβ and VGF;
(ii) contacting said system with a test compound; and
(iii) determining if the test compound alters the level of expression of the gene.

5. The method of claim 3 or 4, wherein the reaction system is a cell-free system.

6. The method of claim 5, wherein the cell-free system is purified protein preparation.

7. The method of claim 5, wherein the cell-free system is a cell-lysate.

8. The method of claim 3 or 4, wherein the reaction system is a whole cell system.

9. The method of claim 3 or 4, wherein the test agent is a organic molecule having a molecular weight less than 2500 amu.

10. The method of claim 3 or 4, which is repeated for a plurality of different test agents.

11. A pharmaceutical preparation comprising, one or more compounds identified in the assay of claim 3 or 4, formulated in a pharmaceutically acceptable excipient.

12. The pharmaceutical preparation of claim 11, further including one or more of a neuronal growth factor, a neuronal survival factor, and a neuronal tropic factor.

13. The pharmaceutical preparation of claim 11, further including an agent that activates CREB-dependent transcription in an amount sufficient to produce a memory enhancing effect.

14. A method for enhancing memory consolidation in an animal, or otherwise enhancing the functional performance of CNS neurons, comprising administering to an animal a pharmaceutical preparation of claim 11.

15. A method for augmenting learning and memory, or otherwise enhancing the functional performance of CNS neurons, comprising administering to an animal a pharmaceutical preparation of claim 11.

16. The method of claim 14 or 15, further comprising administering, conjointly with the pharmaceutical preparation, one or more of a neuronal growth factor, a neuronal survival factor, and a neuronal tropic factor.

17. The method of claim 14 or 15, further comprising administering, conjointly with the pharmaceutical preparation, an agent that activates CREB-dependent transcription in an amount sufficient to produce a memory enhancing effect.

18. The method of claim 17, wherein the CREB activating agent is a cAMP elevating agent.

19. The method of claim 18, wherein at least one cAMP agonist activates adenylate cyclase.

20. The method of claim 17, wherein the CREB activating agent is a cAMP analog.

21. The method of claim 17, wherein the CREB activating agent is a cAMP phosphodiesterase inhibitor.

22. A method for assessing a patient for learning and/or memory functional performance including a step of detecting the expression of a gene selected from the group consisting of of zif 268, insulin-like growth factor, glutamate receptor 1 (GluR1), glutamate receptor 2 (GluR2), c/EBPβ and VGF, or the level of activity of a gene product thereof, in the patient's hippocampus.

23. A method for assessing a patient for learning and/or memory functional performance including a step of detecting the expression of, or a mutation in, one or more genes selected from the group consisting of zif 268, insulin-like growth factor, glutamate receptor 1 (GluR1), glutamate receptor 2 (GluR2), c/EBPβ and VGF, or the level of activity of the gene products thereof, (optionally) in the patient's hippocampus.

24. A method of conducting a drug discovery business comprising:
(i) identifying, by the assay of claim 3 or 4, a test compound which the level of expression of the gene or the activity of the gene product;
(ii) conducting therapeutic profiling of agents identified in step (i), or further analogs thereof, for efficacy and toxicity in animals; and
(iii) formulating a pharmaceutical preparation including one or more agents identified in step (ii) as having an acceptable therapeutic profile.

25. The method of claim 24, including an additional step of establishing a distribution system for distributing the pharmaceutical preparation for sale, and may optionally include establishing a sales group for marketing the pharmaceutical preparation.

26. A method of conducting a target discovery business comprising:
(i) identifying, by the assay of claim 3 or 4, a test compound which the level of expression of the gene or the activity of the gene product;
(ii) (optionally) conducting therapeutic profiling of agents identified in step (i), or further analogs thereof, for efficacy and toxicity in animals; and
(iii). licensing, to a third party, the rights for further drug development of said identified agents.
